SEQUENCE LISTING (4 sequences)

SEQ ID NO: 1 - protein, 477 aa, Kalanchoe blossfeldiana
MGALMDLKWRMRVHGLMGRHLVYKGFILLMAVGVCCVMMSVNFDMNDESSDDGDGGCYLGAELSPFGFRMAVGGVLSHVYALVFPLLGSVRPIICAQNENITLTVFKEIMNEGLLDDGAKAICVGEGSTLAVSALHELGFGNALNVADGGRGRSHHSNSNYVYELDYEDNSFDFVFSRVVDRISVPALLVLELERVLRPGGAGAMIVGSRNLYSGGLIRSATHISAYLKSSDVVNVRAVGSFTLVMFKKRFDKVSAFEHFRLPDICPSVTTNKRIMHYLEPIVRKPHDQSSTSISYVPKFMNISSRGRLVYINIGAGAYVNSSIHKWLKPLYPMRPRAPHVYVIDHNVSVLASYIKSPGVTFIYHPGLAGTGSTDPDRYSSVGEMGERLEDEGFDFVEWFKGTLTPRDFVILAMNAREVELKLLFELYESGAICHVDELFLRCTDGVDCKTPACGDCASLYLGLRNSGVYVHQWFGN

SEQ ID NO: 2 - DNA, 1434 bp, Kalanchoe blossfeldiana
atgggggctttgatggatttgaagtggagaatgagagtgcatgggcttatgggcaggcatttagtctataaaggtttcatcttgctgatggctgtgggggtttgctgtgtgatgatgtctgtgaatttcgatatgaatgatgagagctctgatgatggtgatgggggttgctatctgggggctgagctgagccccttcgggtttcggatggcggttggtggagtgttgagccatgtatatgcacttgtgtttccgctgctaggatcagtcaggccgattatatgtgcgcagaatgagaacatcaccctcaccgtgttcaaggagattatgaatgagggcttgttggatgatggcgccaaggcgatatgcgttggggaaggatccaccttggccgtgtcggccttacacgaattggggttcggaaatgccttaaacgtggcggatggtggaaggggaaggagccatcactcaaactcaaactatgtgtatgagctggattacgaggataacagcttcgatttcgtgttctccagggttgtggacaggatttcggtgcctgcccttttggtgctggagcttgagcgcgtgctgcgcccaggaggggctggagcgatgatcgttgggtcgaggaacttgtactcgggcggattgattcggtccgcgactcacatttccgcatacctgaaaagctctgatgttgtgaatgtgcgcgccgtcgggtcatttactctggttatgttcaagaaaaggttcgataaagtgagcgcctttgagcatttccggctccctgacatctgcccgtccgtcacaaccaacaagcgcatcatgcattatcttgaacccattgtgcggaagccccacgaccaaagctcgacaagcatttcttacgtgcccaagttcatgaatatatcttccaggggccgattggtttacatcaatatcggggcgggcgcatatgtgaactcgagcatacacaagtggctcaagccgttgtacccgatgcgccccagagctcctcacgtgtacgtaatcgatcacaacgtatcggtcctcgcatcctacatcaagtcccccggcgtcaccttcatctaccatcccggtctggctggaacggggtcgacagatccagacaggtactcgtctgtcggagaaatgggtgagcggttggaggatgaagggtttgatttcgtggagtggttcaaagggactctgactcccagagacttcgtcatcctggccatgaatgcgagagaagtggagttgaagcttttgtttgagctgtacgaaagcggagccatatgccatgtggacgagctcttcctccgctgcaccgatggggtggactgcaagaccccggcttgcggggactgcgcgagtctctacctggggcttcggaacagcggtgtgtatgttcatcagtggttcggcaactga

SEQ ID NO: 3 - protein, 477 aa, Kalanchoe blossfeldiana
MGALMDLKWRMRVHGLMGRHLVYKGFILLMAVGVCCVMMSVNFDMNDESSDDGDGGCYLGAELSPFGFRMAVGGVLSHVYALVFPLLGSVRPIICAQNENITLTVFKEIMNEGLLDDGAKAICVGEGSTLAVSALRELGFGNALNVADGGRGRSHHLNSNYVYELDYEDNSFDFVFSRVVDRISVPALLVLELERVLRPGGAGAMIVGSRNLYSGGLIRSATHISAYLKSSDVVNVRAVGSFTLVMFKKRFDKVSAFEHFRLPDVCPSVTTNKRIMHYLEPVVRKPHDQSPTSISYVPKFMNISSRGRLVYINIGAGAYVNSSIHKWLKPLYPMRPRSPHVYVIDHNVSVLASYIKSPGVTFIYHPGLAGTGSTDPDRYASVGEMGERLEDEGFDFVEWFKETLTPRDFVILAMNARAVELKLLFELYESGAICHVDELFLRCSDGVDCKTPACGDCASLYLGLRNSGVYVHQWFGN

SEQ ID NO: 4 - DNA, 1434 bp, Kalanchoe blossfeldiana
atgggggctttgatggatttgaagtggagaatgagagtgcatgggcttatgggcaggcatttagtctataaaggtttcatcttgctgatggctgtgggggtttgctgtgtgatgatgtctgtgaacttcgatatgaatgatgagagctctgatgatggtgatgggggttgctatctgggggctgagctgagccccttcgggtttcggatggcggttggtggagtgttgagccatgtatatgcacttgtgtttccgctgctaggatcagtcaggccgattatatgcgcgcagaatgagaacatcaccctcaccgtgttcaaggagattatgaatgagggcttgttggatgacggcgccaaggcgatatgcgttggggaaggatccaccttggccgtgtccgccttacgcgaattggggtttggaaatgccttaaacgtcgcggatggtggaaggggaaggagccatcacttaaactcaaactatgtgtatgagctggattacgaggataacagcttcgatttcgtgttctccagggttgtggacaggatttcggtgcctgcccttttggtgctggagcttgagcgcgtgctgcgcccaggaggggctggagcgatgatcgttgggtcgaggaacttgtactcggggggattgattcggtccgcaactcacatttccgcatacctgaaaagctccgatgttgtgaatgtgcgcgccgtcgggtcattcactctggtcatgttcaagaaaaggttcgataaagtgagcgcctttgagcatttccggctccctgacgtctgcccgtccgtcacaaccaacaagcgcatcatgcattatcttgaacccgtcgtgcggaagccccacgaccaaagcccgacaagcatttcttacgtgcccaagttcatgaatatatcttccaggggccgattggtttacatcaatatcggcgcgggcgcatatgtgaactcgagcatacacaagtggctcaagccgttgtacccgatgcgccccagatctcctcacgtgtatgtaatcgatcacaacgtatcggtcctcgcatcctacatcaagtcccccggcgtcaccttcatctaccatcccggtctggctggaacggggtcgacagatccagacaggtacgcgtctgtcggagaaatgggtgagcggttggaggatgaagggtttgatttcgtggagtggttcaaagagactctgactcccagagacttcgtcatcctggccatgaatgcgagagccgtggagttgaagcttttgtttgagctgtacgaaagcggagccatatgccatgtggatgagctcttcctccgctgctccgatggggtggactgcaagaccccggcttgcggggactgcgcgagtctctacctggggcttcggaacagcggtgtgtatgttcatcagtggttcggcaactga

(19) United States
(12) Patent Application Publication
(10) Pub. No.: US 2016/0312193 A1
(43) Pub. Date: Oct. 27, 2016

(54) Gene Encoding a Mutant Protein Providing a Decorative Flowering Phenotype in Plants

(71) Applicant: FIDES B.V., De Lier (NL)

(72) Inventors: Izaak Johannes Vlielander, De Lier (NL); Chunting Lang, De Lier (NL); Paulus Cornelis Maris, De Lier (NL); Roger Adrianus Peeters, Weert (NL)

(73) Assignee: Fides BV, De Lier (NL)

(21) Appl. No.: 15/102,254

(22) PCT Filed: Jun. 20, 2014
(86) PCT No.: PCT/EP2014/063019

§ 371 Date: Jun. 6, 2016

(30) Foreign Application Priority Data
Dec. 12, 2013 (EP) PCT/EP2013/076332

(51) Int. Cl. C12N 9/10; C12N 15/82; A01H 5/02

(52) U.S. Cl. CPC C12N 9/1007; A01H 5/0266; C12Y 201/01; C12N 15/827

(57) ABSTRACT

The present invention relates to proteins capable of providing a decorative flowering phenotype in plants and especially in plants belonging to the Kalanchoe genus. The present invention also relates to nucleic acid sequences, or cDNA sequences, and genes encoding the present proteins. The present invention further relates to use of the present proteins, nucleic acid sequences and genes for selecting decorative flowering Kalanchoe plants and decorative flowering Kalanchoe plants comprising the present proteins, mRNA forms of the present cDNAs or the present genes. Specifically, the present invention relates to proteins comprising an amino acid substitution of the amino acid histidine at position 136 and/or the amino acid alanine at position 338 of the methyl transferase protein of a Kalanchoe plant, wherein the amino acid substitution provides a decorative flowering phenotype in the present Kalanchoe plants.

The present invention relates to proteins capable of providing a decorative flowering phenotype to plants and especially to plants belonging to the Kalanchoe genus. The present invention also relates to nucleic acid sequences, or cDNA sequences, and genes encoding the present proteins. The present invention further relates to use of the present proteins, nucleic acid sequences and genes for selecting decorative flowering Kalanchoe plants and to decorative flowering Kalanchoe plants comprising the present proteins, mRNA forms of the present cDNAs or the present genes.

Kalanchoe is a genus of about 125 species of tropical, succulent flowering plants in the family Crassulaceae. Only one species of this genus is known to originate from the Americas, 56 from southern & eastern Africa and 60 species from Madagascar. Kalanchoe plants are also found in south-eastern Asia and China.

Known species belonging to the Kalanchoe genus are Kalanchoe adelae; Kalanchoe arborescens, Kalanchoe beauverdii, Kalanchoe beharensis, Kalanchoe bentii, Kalanchoe blossfeldiana, Kalanchoe bouvetii, Kalanchoe bracteata, Kalanchoe campanulata, Kalanchoe crenata, Kalanchoe crundallii, Kalanchoe daigremontiana, Kalanchoe delagoensis, Kalanchoe dinklagei, Kalanchoe eriophylla, Kalanchoe farinacea, Kalanchoe fedtschenkoi, Kalanchoe figuereidoi, Kalanchoe flammea, Kalanchoe gastonis, Kalanchoe glaucescens, Kalanchoe gracilipes, Kalanchoe grandidieri, Kalanchoe grandiflora, Kalanchoe hildebrantii, Kalanchoe jongmansii, Kalanchoe kewensis, Kalanchoe laciniata, Kalanchoe laetivirens, Kalanchoe lateritia, Kalanchoe laxiflora, Kalanchoe linearifolia, Kalanchoe longiflora, Kalanchoe luciae, Kalanchoe macrochlamys, Kalanchoe manginii, Kalanchoe marnieriana, Kalanchoe marmorata, Kalanchoe millottii, Kalanchoe miniata, Kalanchoe nyikae, Kalanchoe obtusa, Kalanchoe orgyalis, Kalanchoe peltata, Kalanchoe petitiana, Kalanchoe pinnata, Kalanchoe porphyrocalyx, Kalanchoe prolifera, Kalanchoe pubescens, Kalanchoe pumila, Kalanchoe quartiniana, Kalanchoe rhombopilosa, Kalanchoe robusta, Kalanchoe rolandi, Kalanchoe rosei, Kalanchoe rotundifolia, Kalanchoe schizophylla, Kalanchoe serrata, Kalanchoe sexangularis, Kalanchoe streptantha, Kalanchoe suarezensis, Kalanchoe synsepala, Kalanchoe synsepala f. dissecta, Kalanchoe thyrsiflora, Kalanchoe tomentosa, Kalanchoe tubiflora, Kalanchoe uniflora, Kalanchoe velutina and Kalanchoe viguieri.

Most Kalanchoe plants are shrubs or perennial herbaceous plants, but a few are annual or biennial. The largest plant, Kalanchoe beharensis from Madagascar can reach 6 meters but most species are less than 1 meter. Kalanchoe plants are characterized by opening their flowers through growing new cells on the inner surface of the petals to force them outwards and on the outside of the petals to close them.

The Kalanchoe genus was first described by the botanist Michel Adanson in 1763. Reportedly, the name came “from the Chinese name for one of the species.” This Chinese species is thought to have been either Kalanchoe ceratophylla or Kalanchoe spathulata. The genus Bryophyllum was described by Salisbury in 1806 and the genus Kitchingia was created by Baker in 1881. Kitchingia is now regarded as a synonym for Kalanchoe whereas some botanists treat Bryophyllum as a separate genus.

Kalanchoe plants are generally cultivated as ornamental houseplants and rock or succulent garden plants. Kalanchoe plans are popular because of their ease of propagation, low water requirements, and wide variety of flower colors typically borne in clusters well above the vegetative growth. The section Bryophyllum—formerly an independent genus—contains species such as the “Air plant” Kalanchoe pinnata. In these plants, new individuals develop vegetatively as plantlets, also known as bulbils or gemmae, at indents along the leaves. These young plants eventually drop off and root. No males have been found in species of this genus which does flower and produce seeds and is commonly designated as the Mother of Thousands.

In common with other Crassulaceae (such as the genera Tylecodon, Cotyledon and Adromischus), some Kalanchoe species contain bufadienolide cardiac glycosides which can cause cardiac poisoning, particularly in grazing animals. This is a particular problem in the native range of many Kalanchoe species in the Karoo region of South Africa, where the resulting animal disease is known as krimpsiekte (shrinking disease) or cotyledonosis. Similar poisonings have also occurred in Australia.

In traditional medicine, Kalanchoe species have been used to treat ailments such as infections, rheumatism and inflammation. Kalanchoe extracts also have immunosuppressive effects. Kalanchoe pinnata has been recorded in Trinidad and Tobago as being used as a traditional treatment for hypertension.

A variety of bufadienolide compounds have been isolated from various Kalanchoe species. Five different bufadienolides have been isolated from Kalanchoe daigremontiana. Two of these, daigremontianin and bersaldegenin 1,3,5-orthoacetate have been shown to have a pronounced sedative effect. They also have the strong positive inotropic effect associated with cardiac glycosides, and with greater doses an increasing effect on the central nervous system. Bufadienolide compounds isolated from Kalanchoe pinnata include bryophillin A which showed strong anti-tumor promoting activity and bersaldegenin-3-acetate and bryophillin C which were less active. Bryophillin C also showed insecticidal properties.

As indicated, a number of Kalanchoe species, such as K. blossfeldiana, K. laciniata, K. rotundifolia, K. aromatica, K. pubescens, K. grandiflora, K. citrina, K. ambolensis, K. faustii, K. schumacherii, K. pritwitzii, K. flammea, K. figueredoi, K. rauhii, K. obtusa, K. pumila, K. marmorata, K. porphyrocalux, K. jongmansii, K. pinnata, K. diagremontiana, K. gracilipes, K. campanulata, K. latisepela, K. coccinea, K. fedtschenkoi, K. tubiflora, K. decumbens, K. manginii, K. orgyalis, K. crenata, K. tomentosa and hybrids thereof, are cultivated as ornamental houseplants and rock or succulent garden plants. Accordingly, the appearance, and especially the flowering type of these plants is a major factor determining their economic value and use. One flowering type is the so-called decorative flowering type. Kalanchoe plants displaying such decorative flowering type are characterized by having flowers with more than 4 petals per flower, such as at least 6, 8, 10, 15, 25, 35 or more than 40, and substantially all flowers of these Kalanchoe plants display the decorative flowering phenotype.

Although Kalanchoe plants displaying a decorative flowering phenotype are known in the art, there is a need in the art for genetically causal factors of the decorative flowering phenotype, such as genes, thereby not only facilitating and simplifying the breeding of Kalanchoe plants because introgression of the trait can be controlled and selected in relatively early stages of development but also to provide new races of Kalanchoe plants, and especially plants having a mixed genetic make-up of two or more Kalanchoe species, displaying the decorative flowering phenotype.

Accordingly, it is an objective of the present invention, amongst other objectives, to meet the above need in the art, i.e. providing a genetically causal factor for a decorative flowering phenotype.

This objective, amongst other objectives is met according to the present invention by providing proteins, nucleic acid sequences (cDNA or coding sequences) and genes as outlined in the appended claims.

Specifically, this objective, according to a first aspect of the present invention, is met by proteins comprising an amino acid substitution of the amino acid histidine at position 136 and the amino acid alanine at position 338 of the methyltransferase protein of a Kalanchoe plant whereby the present amino acid substitutions provide a decorative flowering phenotype in the present Kalanchoe plant.

Specifically, this objective, according to a first aspect of the present invention, is met by proteins comprising an amino acid substitution of the amino acid histidine at position 136 of the methyltransferase protein of a Kalanchoe plant, whereby the present amino acid substitution provides a decorative flowering phenotype in said the present Kalanchoe plant.

Specifically, this objective, according to a first aspect of the present invention, is met by proteins comprising an amino acid substitution of the amino acid alanine at position 338 of the methyltransferase protein of a Kalanchoe plant, whereby the present amino acid substitution provides a decorative flowering phenotype in the present Kalanchoe plant.

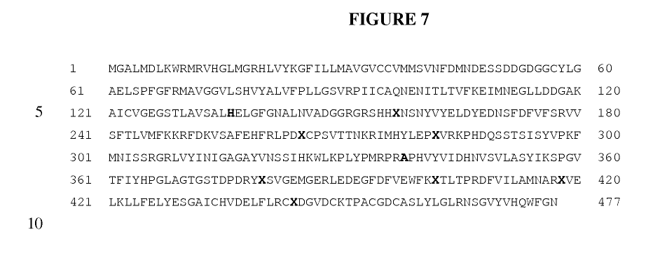

According to a preferred embodiment of this first aspect, the present invention relates to proteins comprising the amino acid sequence as depicted in FIG. 7 with an amino acid substitution of the amino acid histidine at position 136 and the amino acid alanine at position 338 and proteins having at least at least 70%, preferably at least 85%, more preferably at least 90%, even more preferably at least 95% and most preferably 99% sequence identity therewith under the condition that the proteins have methyltransferase activity.

According to a preferred embodiment of this first aspect, the present invention relates to proteins comprising the amino acid sequence as depicted in FIG. 7 with an amino acid substitution of the amino acid histidine at position 136 and proteins having at least at least 70%, preferably at least 85%, more preferably at least 90%, even more preferably at least 95% and most preferably 99% sequence identity therewith under the condition that the proteins have methyltransferase activity.

According to a preferred embodiment of this first aspect, the present invention relates to proteins comprising the amino acid sequence as depicted in FIG. 7 with an amino acid substitution of the amino acid alanine at position 338 and proteins having at least at least 70%, preferably at least 85%, more preferably at least 90%, even more preferably at least 95% and most preferably 99% sequence identity therewith under the condition that the proteins have methyltransferase activity.

Within the context of the present invention, homologous proteins are considered under the condition that these proteins display at least 70%, preferably at least 85%, more preferably at least 90%, even more preferably at least 95% and most preferably 99% sequence identity over the entire length with the protein sequence as shown in FIG. 7 and under the condition that at least a position corresponding with position 136 or 338 of the protein sequence shown in FIG. 7 is mutated.

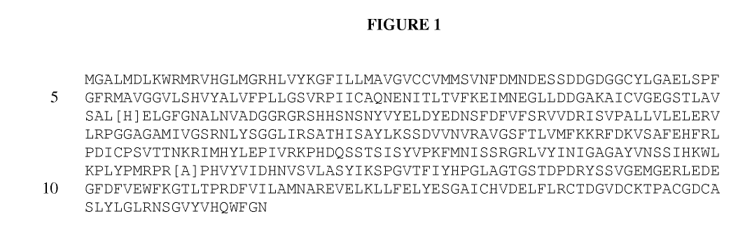

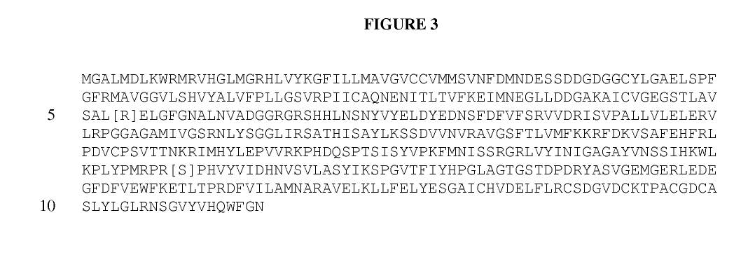

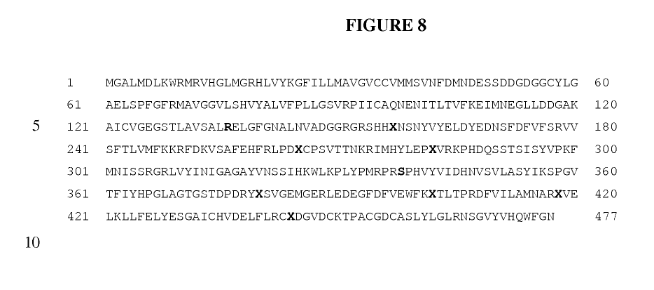

The amino acid sequences shown in FIG. 1 and FIG. 7 are the amino acid sequences of proteins not providing the decorative flowering phenotype and the amino acid sequences shown in FIG. 3 and FIG. 8 are the amino acid sequences of a proteins providing the decorative flowering phenotype through a mutation, i.e. in the present case (an) amino acid substitution(s). In FIGS. 3 and 8, amino acid substitutions, or mutations, are shown but also deletions, truncations and amino acid insertions are contemplated within the context of the present invention.

The present decorative flowering phenotype can be provided by a single gene mutation, i.e. an amino acid substitution at position 136 or 338 or amino acid substitutions at positions 136 and 338, in de coding sequence resulting in a mutated protein. Inheritance experiments have shown that the mutation is dominant, i.e. both for the mutation(s) homozygous and heterozygous Kalanchoe plants display the decorative flowering phenotype however heterozygous Kalanchoe plants are preferred.

According to a more preferred embodiment of this first aspect of the present invention, the present proteins comprise an alanine (A or Ala) to serine (S or Ser) amino acid substitution at position 338 and an histidine (H or His) to arginine (R or Arg) amino acid substitution at position 136 although replacements with other amino acids are contemplated within the context of the present invention such as replacements with cysteine, aspartic acid, glutamic acid, phenylalanine, glycine, histidine, isoleucine, lysine, leucine, methionine, asparagine, proline, glutamine, arginine, threonine, valine, tryptophan and tyrosine.

According to a more preferred embodiment of this first aspect of the present invention, the present proteins comprise an alanine (A or Ala) to serine (S or Ser) amino acid substitution at position 338 although replacements with other amino acids are contemplated within the context of the present invention such as replacements with cysteine, aspartic acid, glutamic acid, phenylalanine, glycine, histidine, isoleucine, lysine, leucine, methionine, asparagine, proline, glutamine, arginine, threonine, valine, tryptophan and tyrosine.

According to a more preferred embodiment of this first aspect of the present invention, the present proteins comprise an histidine (H or His) to arginine (R or Arg) amino acid substitution at position 136 although replacements with other amino acids are contemplated within the context of the present invention such as replacements with cysteine, aspartic acid, glutamic acid, phenylalanine, glycine, histidine, isoleucine, lysine, leucine, methionine, asparagine, proline, glutamine, arginine, threonine, valine, tryptophan and tyrosine.

According to an even more preferred embodiment of this first aspect of the invention, the present proteins comprise the amino acid sequences as shown in FIG. 3 or 8. Formulated differently, the present proteins have substantially the amino acid sequences as shown in FIG. 3 or 8.

According to a second aspect of the present invention, the proteins comprise at least one mutation in the amino acid sequence as shown in FIG. 1 wherein the at least one mutation provides a decorative flowering phenotype in Kalanchoe plants. Having the knowledge that the proteins shown in FIG. 1 are a causative factor for the decorative flowering phenotype, the skilled person, using standard means and methods, is readily able to mutate this protein, for example by EMS or site directed mutagenesis followed by an appropriate selection, for example by sequencing or PCR, thereby providing Kalanchoe plants showing the decorative flowering phenotype. As an alternative, the present genes can be introduced in a breeding germplasm by conventional breeding.

According to an especially preferred embodiment of these first and second aspects of the present invention, the proteins disclosed are obtained, or originate, from a decorative flowering Kalanchoe blossfeldiana plant.

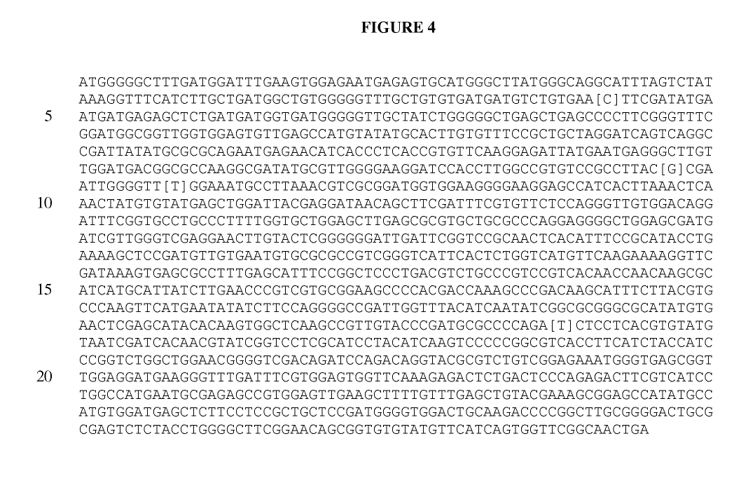

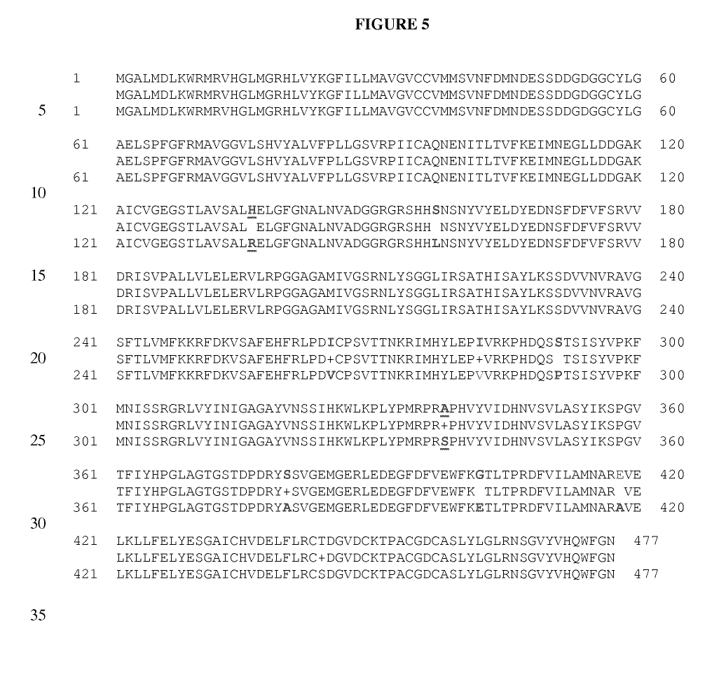

According to a third aspect, the present invention relates to nucleic acid sequences encoding the present proteins, preferably substantially the nucleic acid sequence as shown in FIG. 4.

According to a fourth aspect, the present invention relates to genes capable of being transcribed into the present nucleic acid sequences. Formulated differently, the present invention encompasses genes capable of, under appropriate conditions, being a template for transcribing mRNA which mRNA is subsequently translated into the present protein sequences.

According to a fifth aspect, the present invention relates to the use of the present proteins, the present nucleic acid sequences or the present genes for selecting decorative flowering Kalanchoe plants. Such selection can comprise identification, preferably in an early stage of development, of decorative flowering Kalanchoe plants by, for example, hybridization, PCR, ELISA, restriction analysis or Northern/Southern blots thereby facilitating the breeding process of existing and new Kalanchoe plants.

The present use is preferably used for the selection of decorative flowering Kalanchoe plants selected from the group consisting of Kalanchoe adelae, Kalanchoe arborescens, Kalanchoe beauverdii, Kalanchoe beharensis, Kalanchoe bentii, Kalanchoe blossfeldiana, Kalanchoe bouvetii, Kalanchoe bracteata, Kalanchoe campanulata, Kalanchoe crenata, Kalanchoe crundallii, Kalanchoe daigremontiana, Kalanchoe delagoensis, Kalanchoe dinklagei, Kalanchoe eriophylla, Kalanchoe farinacea, Kalanchoe fedtschenkoi, Kalanchoe figuereidoi, Kalanchoe flammea, Kalanchoe gastonis, Kalanchoe glaucescens, Kalanchoe gracilipes, Kalanchoe grandidieri, Kalanchoe grandiflora, Kalanchoe hildebrantii, Kalanchoe jongmansii, Kalanchoe kewensis, Kalanchoe laciniata, Kalanchoe laetivirens, Kalanchoe lateritia, Kalanchoe laxiflora, Kalanchoe linearifolia, Kalanchoe longiflora, Kalanchoe luciae, Kalanchoe macrochlamys, Kalanchoe manginii, Kalanchoe marnieriana, Kalanchoe marmorata, Kalanchoe millottii, Kalanchoe miniata, Kalanchoe nyikae, Kalanchoe obtusa, Kalanchoe orgyalis, Kalanchoe peltata, Kalanchoe petitiana, Kalanchoe pinnata, Kalanchoe porphyrocalyx, Kalanchoe prolifera, Kalanchoe pubescens, Kalanchoe pumila, Kalanchoe quartiniana, Kalanchoe rhombopilosa, Kalanchoe robusta, Kalanchoe rolandi, Kalanchoe rosei, Kalanchoe rotundifolia, Kalanchoe schizophylla, Kalanchoe serrata, Kalanchoe sexangularis, Kalanchoe streptantha, Kalanchoe suarezensis, Kalanchoe synsepala, Kalanchoe synsepala f. dissecta, Kalanchoe thyrsiflora, Kalanchoe tomentosa, Kalanchoe tubiflora, Kalanchoe uniflora, Kalanchoe velutina and Kalanchoe viguieri; more preferably decorative flowering Kalanchoe plants selected from the group consisting of K. blossfeldiana, K. laciniata, K. rotundifolia, K. aromatica, K. pubescens, K. grandiflora, K. citrina, K. ambolensis, K. faustii, K. schumacherii, K. pritwitzii, K. flammea, K. figueredoi, K. rauhii, K. obtusa, K. pumila, K. marmorata, K. porphyrocalux, K. jongmansii, K. pinnata, K. diagremontiana, K. gracilipes, K. campanulata, K. latisepela, K. coccinea, K. fedtschenkoi, K. tubiflora, K. decumbens, K. manginii, K. orgyalis, K. crenata, K. tomentosa and hybrids thereof.

According to a sixth and seventh aspect, the present invention relates to plants or plant parts comprising the present proteins, the present nucleic acid sequences (in the form of mRNA) or the present genes and as well as to plants obtainable or obtained through the use of the present proteins, the present nucleic acid sequences or the present genes.

According to a preferred embodiment of this sixth and seventh aspect of the present invention, the plant is selected from the group consisting of Kalanchoe adelae, Kalanchoe arborescens, Kalanchoe beauverdii, Kalanchoe beharensis, Kalanchoe bentii, Kalanchoe blossfeldiana, Kalanchoe bouvetii, Kalanchoe bracteata, Kalanchoe campanulata, Kalanchoe crenata, Kalanchoe crundallii, Kalanchoe daigremontiana, Kalanchoe delagoensis, Kalanchoe dinklagei, Kalanchoe eriophylla, Kalanchoe farinacea, Kalanchoe fedtschenkoi, Kalanchoe figuereidoi, Kalanchoe flammea, Kalanchoe gastonis, Kalanchoe glaucescens, Kalanchoe gracilipes, Kalanchoe grandidieri, Kalanchoe grandiflora, Kalanchoe hildebrantii, Kalanchoe jongmansii, Kalanchoe kewensis, Kalanchoe laciniata, Kalanchoe laetivirens, Kalanchoe lateritia, Kalanchoe laxiflora, Kalanchoe linearifolia, Kalanchoe longiflora, Kalanchoe luciae, Kalanchoe macrochlamys, Kalanchoe manginii, Kalanchoe marnieriana, Kalanchoe marmorata, Kalanchoe millottii, Kalanchoe miniata, Kalanchoe nyikae, Kalanchoe obtusa, Kalanchoe orgyalis, Kalanchoe peltata, Kalanchoe petitiana, Kalanchoe pinnata, Kalanchoe porphyrocalyx, Kalanchoe prolifera, Kalanchoe pubescens, Kalanchoe pumila, Kalanchoe quartiniana, Kalanchoe rhombopilosa, Kalanchoe robusta, Kalanchoe rolandi, Kalanchoe rosei, Kalanchoe rotundifolia, Kalanchoe schizophylla, Kalanchoe serrata, Kalanchoe sexangularis, Kalanchoe streptantha, Kalanchoe suarezensis, Kalanchoe synsepala, Kalanchoe synsepala f. dissecta, Kalanchoe thyrsiflora, Kalanchoe tomentosa, Kalanchoe tubiflora, Kalanchoe uniflora, Kalanchoe velutina and Kalanchoe viguieri; more preferably decorative flowering Kalanchoe plants selected from the group consisting of K. blossfeldiana, K. laciniata, K. rotundifolia, K. aromatica, K. pubescens, K. grandiflora, K. citrina, K. ambolensis, K. faustii, K. schumacherii, K. pritwitzii, K. flammea, K. figueredoi, K. rauhii, K. obtusa, K. pumila, K. marmorata, K. porphyrocalux, K. jongmansii, K. pinnata, K. diagremontiana, K. gracilipes, K. campanulata, K. latisepela, K. coccinea, K. fedtschenkoi, K. tubiflora, K. decumbens, K. manginii, K. orgyalis, K. crenata, K. tomentosa and hybrids thereof.

Throughout the description and claims, reference is made to figures wherein:

FIG. 1: shows the amino acid sequence of a protein not causing a decorative flowering phenotype in Kalanchoe

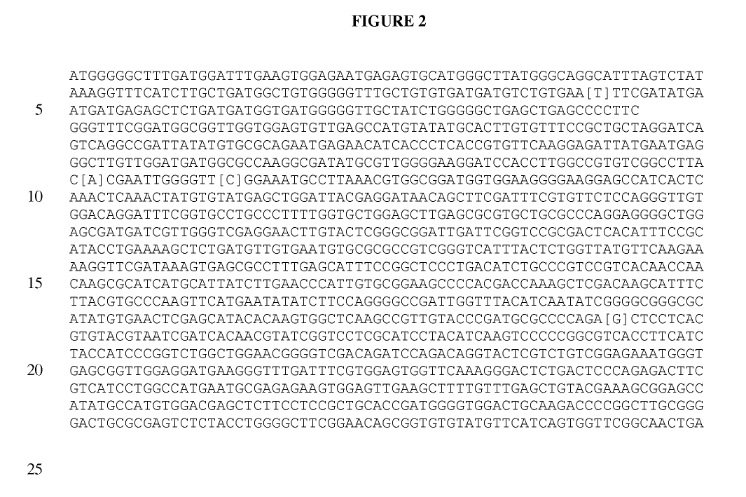

FIG. 2: shows the cDNA sequence encoding the amino acid sequence shown in FIG. 1;

FIG. 3: shows the amino acid sequence of a protein causing a decorative flowering phenotype in Kalanchoe;

FIG. 4: shows the cDNA sequence encoding the amino acid sequence shown in FIG. 3;

FIG. 5: shows an alignment of the amino acid sequences shown in FIGS. 1 and 3;

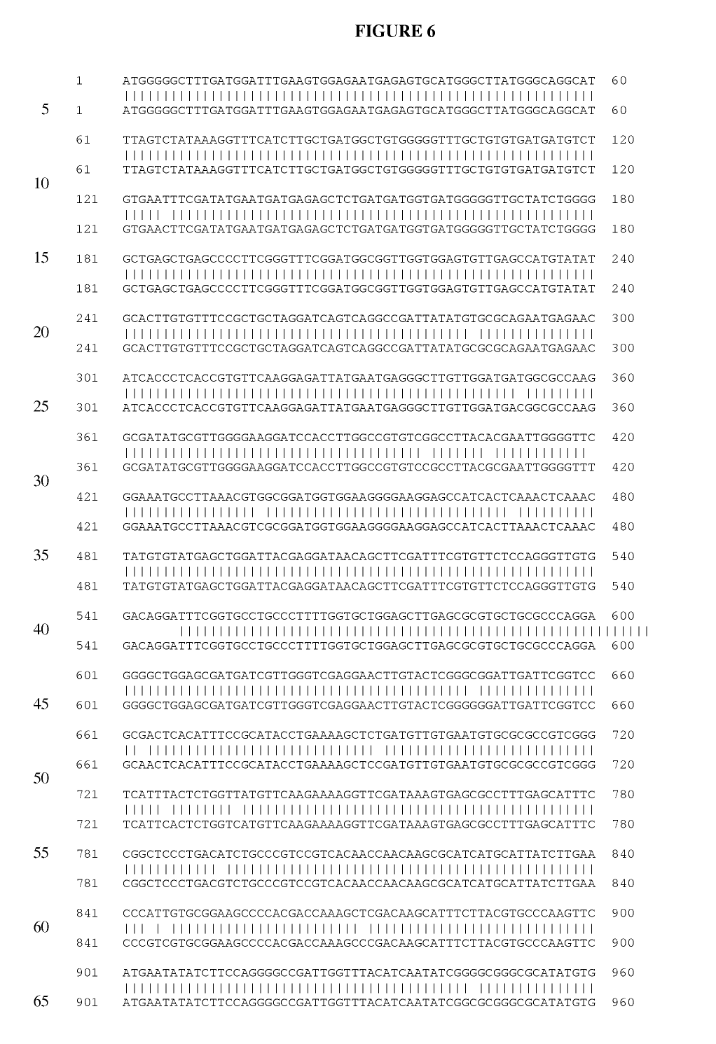

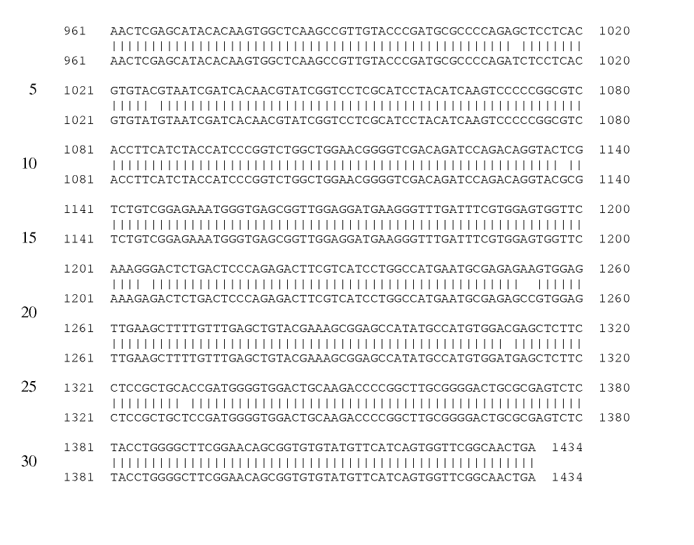

FIG. 6: shows an alignment of the nucleotide sequences shown in FIGS. 2 and 4;

FIG. 7: shows variable positions denoted with an X (any naturally occurring amino acid) in the amino acid sequence of a protein not causing a decorative flowering phenotype in Kalanchoe;

FIG. 8: shows variable positions denoted with an X (any naturally occurring amino acid) in the amino acid sequence of a protein causing a decorative flowering phenotype in Kalanchoe.

### EXAMPLE

The presence of the present methyltransferases as depicted in FIGS. 7 and 8 was determined in several Kalanchoe species having a decorative flowering phenotype. As controls, several non-flowering phenotype Kalanchoe species were included. The results are summarized in Table 1 below.

TABLE 1

| | Decorative | Methyltransferase | Methyltransferase |
|---|---|---|---|
| Kalanchoe | flowering | protein as depicted | protein as depicted |
| variant | phenotype | in FIG. 7 | in FIG. 8 |
| Tylo | Yes | Yes | Yes |
| Taylor | Yes | Yes | Yes |
| Jodie | Yes | Yes | Yes |
| African Sunshine | Yes | Yes | Yes |
| Mercedes | Yes | Yes | Yes |
| Don Antonio | Yes | Yes | Yes |
| 12-016-02 | Yes | Yes | Yes |
| Foster | Yes | Yes | Yes |
| Paris | Yes | Yes | Yes |
| Leonardo | Yes | Yes | Yes |
| Alexandra | No | Yes | No |
| Milos | No | Yes | No |
| Snowdon | No | Yes | No |
| Josefine Yellow | No | Yes | No |
| Molly | No | Yes | No |
| African Love | No | Yes | No |
| Amora | No | Yes | No |
| Catalana | No | Yes | No |
| Venetia | No | Yes | No |

As shown in Table 1, all decorative flowering Kalanchoe species comprised besides a wild-type methyltransferase protein a mutated methyltransferase protein with amino acid substitutions at positions 136 and 338.

1. A protein comprising an amino acid substitution of the amino acid histidine at position 136 and/or the amino acid alanine at position 338 of the methyltransferase protein of a plant, said amino acid substitution provides a decorative flowering phenotype in said plant.

2. The protein according to claim 1, wherein said plant is selected from the group consisting of Kalanchoe adelae, Kalanchoe arborescens, Kalanchoe beauverdii, Kalanchoe beharensis, Kalanchoe bentii, Kalanchoe blossfeldiana, Kalanchoe bouvetii, Kalanchoe bracteata, Kalanchoe campanulata, Kalanchoe crenata, Kalanchoe crundallii, Kalanchoe daigremontiana, Kalanchoe delagoensis, Kalanchoe dinklagei, Kalanchoe eriophylla, Kalanchoe farinacea, Kalanchoe fedtschenkoi, Kalanchoe figuereidoi, Kalanchoe flammea, Kalanchoe gastonis, Kalanchoe glaucescens, Kalanchoe gracilipes, Kalanchoe grandidieri, Kalanchoe grandiflora, Kalanchoe hildebrantii, Kalanchoe jongmansii, Kalanchoe kewensis, Kalanchoe laciniata, Kalanchoe laetivirens, Kalanchoe lateritia, Kalanchoe laxiflora, Kalanchoe linearifolia, Kalanchoe longiflora, Kalanchoe luciae, Kalanchoe macrochlamys, Kalanchoe manginii, Kalanchoe marnieriana, Kalanchoe marmorata, Kalanchoe millottii, Kalanchoe miniata, Kalanchoe nyikae, Kalanchoe obtusa, Kalanchoe orgyalis, Kalanchoe peltata, Kalanchoe petitiana, Kalanchoe pinnata, Kalanchoe porphyrocalyx, Kalanchoe prolifera, Kalanchoe pubescens, Kalanchoe pumila, Kalanchoe quartiniana, Kalanchoe rhombopilosa, Kalanchoe robusta, Kalanchoe rolandi, Kalanchoe rosei, Kalanchoe rotundifolia, Kalanchoe schizophylla, Kalanchoe serrata, Kalanchoe sexangularis, Kalanchoe streptantha, Kalanchoe suarezensis, Kalanchoe synsepala, Kalanchoe synsepala f. dissecta, Kalanchoe thyrsiflora, Kalanchoe tomentosa, Kalanchoe tubiflora, Kalanchoe uniflora, Kalanchoe velutina and Kalanchoe viguieri; preferably decorative flowering Kalanchoe plants selected from the group consisting of K. blossfeldiana, K. laciniata, K. rotundifolia, K. aromatica, K. pubescens, K. grandiflora, K. citrina, K. ambolensis, K. faustii, K. schumacherii, K. pritwitzii, K. flammea, K. figueredoi, K. rauhii, K. obtusa, K. pumila, K. marmorata, K. porphyrocalux, K. jongmansii, K. pinnata, K. diagremontiana, K. gracilipes, K. campanulata, K. latisepela, K. coccinea, K. fedtschenkoi, K. tubiflora, K. decumbens, K. manginii, K. orgyalis, K. crenata, K. tomentosa and hybrids thereof.

3. The protein according to claim 1, wherein said protein comprises the amino acid sequence as depicted in FIG. 7 with an amino acid substitution of the amino acid histidine at position 136 and/or the amino acid alanine at position 338 and proteins having at least 70%, preferably at least 85%, more preferably at least 90%, even more preferably at least 95% and most preferably at least 99% sequence identity and having a methyltransferase activity.

4. The protein according to claim 1, wherein said amino acid substitution at position 136 is a substitution of the amino acid histidine with the amino acid arginine.

5. The protein according to claim 1, wherein said amino acid substitution at position 338 is a substitution of the amino acid alanine with the amino acid serine.

6. The protein according to claim 1, wherein said protein comprises the amino acid sequence as shown in FIG. 8.

7. The protein according to claim 6, wherein said protein comprises the amino acid sequence as shown in FIG. 3.

8. The protein according to claim 1, wherein said decorative flowering phenotype is characterized by flowers with more than 4 petals per flower.

9. The protein according to claim 1, wherein said protein is obtained, or originates, from a decorative flowering Kalanchoe blossfeldiana plant.

10. A nucleic acid sequence encoding a protein according to claim 1.

11. A nucleic acid sequence as shown in FIG. 4.

12. A gene encoding a protein according to claim 1.

13. Use of a protein according to claim 1 for selecting decorative flowering Kalanchoe plants.

14. The use according to claim 13, wherein said decorative flowering Kalanchoe plants are selected from the group consisting of Kalanchoe adelae, Kalanchoe arborescens, Kalanchoe beauverdii, Kalanchoe beharensis, Kalanchoe bentii, Kalanchoe blossfeldiana, Kalanchoe bouvetii, Kalanchoe bracteata, Kalanchoe campanulata, Kalanchoe crenata, Kalanchoe crundallii, Kalanchoe daigremontiana, Kalanchoe delagoensis, Kalanchoe dinklagei, Kalanchoe eriophylla, Kalanchoe farinacea, Kalanchoe fedtschenkoi, Kalanchoe figuereidoi, Kalanchoe flammea, Kalanchoe gastonis, Kalanchoe glaucescens, Kalanchoe gracilipes, Kalanchoe grandidieri, Kalanchoe grandiflora, Kalanchoe hildebrantii, Kalanchoe jongmansii, Kalanchoe kewensis, Kalanchoe laciniata, Kalanchoe laetivirens, Kalanchoe lateritia, Kalanchoe laxiflora, Kalanchoe linearifolia, Kalanchoe longiflora, Kalanchoe luciae, Kalanchoe macrochlamys, Kalanchoe manginii, Kalanchoe marnieriana, Kalanchoe marmorata, Kalanchoe millottii, Kalanchoe miniata, Kalanchoe nyikae, Kalanchoe obtusa, Kalanchoe orgyalis, Kalanchoe peltata, Kalanchoe petitiana, Kalanchoe pinnata, Kalanchoe porphyrocalyx, Kalanchoe prolifera, Kalanchoe pubescens, Kalanchoe pumila, Kalanchoe quartiniana, Kalanchoe rhombopilosa, Kalanchoe robusta, Kalanchoe rolandi, Kalanchoe rosei, Kalanchoe rotundifolia, Kalanchoe schizophylla, Kalanchoe serrata, Kalanchoe sexangularis, Kalanchoe streptantha, Kalanchoe suarezensis, Kalanchoe synsepala, Kalanchoe synsepala f. dissecta, Kalanchoe thyrsiflora, Kalanchoe tomentosa, Kalanchoe tubiflora, Kalanchoe uniflora, Kalanchoe velutina and Kalanchoe viguieri; preferably decorative flowering Kalanchoe plants selected from the group consisting of K. blossfeldiana, K. laciniata, K. rotundifolia, K. aromatica, K. pubescens, K. grandiflora, K. citrina, K. ambolensis, K. faustii, K. schumacherii, K. pritwitzii, K. flammea, K. figueredoi, K. rauhii, K. obtusa, K. pumila, K. marmorata, K. porphyrocalux, K. jongmansii, K. pinnata, K. diagremontiana, K. gracilipes, K. campanulata, K. latisepela, K. coccinea, K. fedtschenkoi, K. tubiflora, K. decumbens, K. manginii, K. orgyalis, K. crenata, K. tomentosa and hybrids thereof.

15. Plant, protoplast or plant parts comprising a protein according to claim 1.

16. The plant, protoplast or plant parts according to claim 15, wherein said plant, protoplast or plant parts are selected from the group consisting of Kalanchoe adelae, Kalanchoe arborescens, Kalanchoe beauverdii, Kalanchoe beharensis, Kalanchoe bentii, Kalanchoe blossfeldiana, Kalanchoe bouvetii, Kalanchoe bracteata, Kalanchoe campanulata, Kalanchoe crenata, Kalanchoe crundallii, Kalanchoe daigremontiana, Kalanchoe delagoensis, Kalanchoe dinklagei, Kalanchoe eriophylla, Kalanchoe farinacea, Kalanchoe fedtschenkoi, Kalanchoe figuereidoi, Kalanchoe flammea, Kalanchoe gastonis, Kalanchoe glaucescens, Kalanchoe gracilipes, Kalanchoe grandidieri, Kalanchoe grandiflora, Kalanchoe hildebrantii, Kalanchoe jongmansii, Kalanchoe kewensis, Kalanchoe laciniata, Kalanchoe laetivirens, Kalanchoe lateritia, Kalanchoe laxiflora, Kalanchoe linearifolia, Kalanchoe longiflora, Kalanchoe luciae, Kalanchoe macrochlamys, Kalanchoe manginii, Kalanchoe marnieriana, Kalanchoe marmorata, Kalanchoe millottii, Kalanchoe miniata, Kalanchoe nyikae, Kalanchoe obtusa, Kalanchoe orgyalis, Kalanchoe peltata, Kalanchoe petitiana, Kalanchoe pinnata, Kalanchoe porphyrocalyx, Kalanchoe prolifera, Kalanchoe pubescens, Kalanchoe pumila, Kalanchoe quartiniana, Kalanchoe rhombopilosa, Kalanchoe robusta, Kalanchoe rolandi, Kalanchoe rosei, Kalanchoe rotundifolia, Kalanchoe schizophylla, Kalanchoe serrata, Kalanchoe sexangularis, Kalanchoe streptantha, Kalanchoe suarezensis, Kalanchoe synsepala, Kalanchoe synsepala f. dissecta, Kalanchoe thyrsiflora, Kalanchoe tomentosa, Kalanchoe tubiflora, Kalanchoe uniflora, Kalanchoe velutina and Kalanchoe viguieri; preferably decorative flowering Kalanchoe plants selected from the group consisting of K. blossfeldiana, K. laciniata, K. rotundifolia, K. aromatica, K. pubescens, K. grandiflora, K. citrina, K. ambolensis, K. faustii, K. schumacherii, K. pritwitzii, K. flammea, K. figueredoi, K. rauhii, K. obtusa, K. pumila, K. marmorata, K. porphyrocalux, K. jongmansii, K. pinnata, K. diagremontiana, K. gracilipes, K. campanulata, K. latisepela, K. coccinea, K. fedtschenkoi, K. tubiflora, K. decumbens, K. manginii, K. orgyalis, K. crenata, K. tomentosa and hybrids thereof.

17. Use of a protein according to claim 1 for development of a molecular marker indicative of, or associated with, a decorative flowering phenotype.

18. A plant, protoplast or plant parts comprising a mutated methyltransferase providing a decorative phenotype said methyltransferase is mutated as compared to the methyltransferase as shown in FIG. 1 or FIG. 7.

19. The plant, protoplast or plant parts according to claim 18, wherein said plant, protoplast or plant parts are selected from the group consisting of Kalanchoe adelae, Kalanchoe arborescens, Kalanchoe beauverdii, Kalanchoe beharensis, Kalanchoe bentii, Kalanchoe blossfeldiana, Kalanchoe bouvetii, Kalanchoe bracteata, Kalanchoe campanulata, Kalanchoe crenata, Kalanchoe crundallii, Kalanchoe daigremontiana, Kalanchoe delagoensis, Kalanchoe dinklagei, Kalanchoe eriophylla, Kalanchoe farinacea, Kalanchoe fedtschenkoi, Kalanchoe figuereidoi, Kalanchoe flammea, Kalanchoe gastonis, Kalanchoe glaucescens, Kalanchoe gracilipes, Kalanchoe grandidieri, Kalanchoe grandiflora, Kalanchoe hildebrantii, Kalanchoe jongmansii, Kalanchoe kewensis, Kalanchoe laciniata, Kalanchoe laetivirens, Kalanchoe lateritia, Kalanchoe laxiflora, Kalanchoe linearifolia, Kalanchoe longiflora, Kalanchoe luciae, Kalanchoe macrochlamys, Kalanchoe manginii, Kalanchoe marnieriana, Kalanchoe marmorata, Kalanchoe millottii, Kalanchoe miniata, Kalanchoe nyikae, Kalanchoe obtusa, Kalanchoe orgyalis, Kalanchoe peltata, Kalanchoe petitiana, Kalanchoe pinnata, Kalanchoe porphyrocalyx, Kalanchoe prolifera, Kalanchoe pubescens, Kalanchoe pumila, Kalanchoe quartiniana, Kalanchoe rhombopilosa, Kalanchoe robusta, Kalanchoe rolandi, Kalanchoe rosei, Kalanchoe rotundifolia, Kalanchoe schizophylla, Kalanchoe serrata, Kalanchoe sexangularis, Kalanchoe streptantha, Kalanchoe suarezensis, Kalanchoe synsepala, Kalanchoe synsepala f. dissecta, Kalanchoe thyrsiflora, Kalanchoe tomentosa, Kalanchoe tubiflora, Kalanchoe uniflora, Kalanchoe velutinaand Kalanchoe viguieri; preferably decorative flowering Kalanchoe plants selected from the group consisting of K. blossfeldiana, K. laciniata, K. rotundifolia, K. aromatica, K. pubescens, K. grandiflora, K. citrina, K. ambolensis, K. faustii, K. schumacherii, K. pritwitzii, K. flammea, K. figueredoi, K. rauhii, K. obtusa, K. pumila, K. marmorata, K. porphyrocalux, K. jongmansii, K. pinnata, K. diagremontiana, K. gracilipes, K. campanulata, K. latisepela, K. coccinea, K. fedtschenkoi, K. tubiflora, K. decumbens, K. manginii, K. orgyalis, K. crenata, K. tomentosa and hybrids thereof.
